(19) United States
(12) Patent Application Publication
(10) Pub. No.: US 2011/0144630 A1
(43) Pub. Date: Jun. 16, 2011

(54) Fiber optic device with controlled reuse

(76) Inventor: Marvin P. Loeb, Laguna Woods, CA (US)

(21) Appl. No.: 12/653,309

(22) Filed: Dec. 10, 2009

(51) Int. Cl. A61B 18/22

(52) U.S. Cl. 606/16

(57) ABSTRACT

A fiber optic device includes an optical fiber passing through a handpiece that defines an internal passageway with an apertured endwall at a distal end of the passageway. A portion of the optical fiber is disposed within the passageway, and a stop member, such as a rigid sheath, within the passageway is attached to the optical fiber. An indexing member releasably engages the rigid sheath to maintain the rigid sheath at a predetermined position within the passageway. The rigid sheath preferably contains a series of index positions for incrementally adjusting the position of the sheath within the passageway. The rigid sheath is slidably mounted in the passageway and is configured to interfere with the endwall when the rigid sheath is released for movement within the internal cavity to its final position within the passageway.

### FIELD OF THE INVENTION

The present invention relates to fiber optic devices, whose frequency of use can be controlled, and especially to such devices suitable for use in a medical procedure.

### BACKGROUND OF THE INVENTION

Optical fibers, sometimes called fiber-optics, are used to transmit laser energy in a wide variety of medical and surgical procedures to vaporize, cut, coagulate, shrink and denature tissue. The ability to create a variety of tissue effects by varying the amount or wavelength of the laser energy being delivered makes optical fibers a desirable means for delivering laser energy through body orifices or tiny punctures, instead of larger incisions, which are necessary for the use of laser energy not transmittable through optical fibers or other surgical tools.

Optical fibers typically consist of a fused silica or quartz core, which is covered by a thin glass cladding, which is further encased by a relatively thicker buffer coating of a fluorocarbon or a similar plastic to prevent mechanical damage to the optical fiber.

Laser energy reflected from the tissue can vaporize the protective buffer coating of the optical fiber, however, leaving the optical fiber subject to fracture. Also, the distal end portion of the optical fiber can be deformed or melted by laser energy reflected from the target tissue, causing laser energy to be emitted in an undesired direction, perhaps affecting an unintended tissue, nerve or blood vessel.

One solution is to periodically clip-off a portion of buffer coating from the distal end of the optical fiber. For example, about 4 to 12 mm of the distal end of the buffer coating of the optical fiber, preferably about 8 mm, is clipped off with a clipping tool, leaving 8 mm of bared optical fiber exposed. Then about 2 to 6 mm of the exposed bared distal end of the optical fiber, preferably about 4 mm, is cleaved-off with a clipping tool. This process is called “clipping and cleaving,” after which the fiber-optic device can be cleaned and sterilized for another use. Clipping and cleaving tools are often provided or sold to users of lasers by the optical fiber manufacturer.

Since fiber-optic devices are usually supplied in lengths of about 2.5 to 3 meters, they can be clipped and cleaved up to 100 or more times before the laser source, which cannot enter the sterile field in the operating room, can no longer be moved closer and the optical fiber cannot be easily handled during use.

Excessive reuse of fiber-optic devices is not always desirable. In some instances, as known in the art, a microchip-bearing or bar-coded card is supplied with each optical fiber. Alternatively, as known in the art, a microchip may be embedded in the connector of the optical fiber device. In either case, when the microchip-bearing or bar-coded card is inserted into a card reader or the connector is inserted into the optical coupler of the laser, it is recognized by the computer's microprocessor. If the card or connector has been recognized before, the microprocessor will not allow the laser to be turned-on.

The laser's microprocessor can be programmed to permit, for example, the optical fiber to be used one, five, ten or any number of times. However, if a hospital, surgery center or physician already owns a laser without such a microprocessor, the laser cannot be refitted with such a microprocessor without the customer's consent, which is unlikely to be given. Since thousands of lasers without such microprocessors have been sold to hospitals, surgery centers and physicians, there is no way to limit the number of reuses of conventional optical fibers with these lasers.

Customers sometimes leave a laser unit with such a microprocessor on “standby” between medical procedures or surgeries, during which it is operating at a very low level and its energy delivery port is closed. Initially, an unused fiber-optic device can be connected, recognized and the laser “enabled”. When the medical procedure or surgery is completed, the laser remains on standby, the optical fiber remains connected to the laser, and the distal end portion of the optical fiber can be cleaned, sterilized and reused. However, if left on standby all day or through one or more nights, the laser's lifetime may be shortened.

It would be desirable to satisfy the manufacturer's desire to prevent excessive reuse of fiber-optic devices by the operators of existing lasers in hospitals, surgery centers or physicians' offices, while satisfying the customer's desire to amortize the cost of the fiber-optic device over a sufficient number of medical procedures or surgeries, to reduce the cost per case. This is particularly important in countries where governmental organizations or insurance plans do not pay hospitals, surgery centers or physicians for medical procedures as handsomely as Medicare and insurance plans in the United States.

### SUMMARY OF THE INVENTION

The present invention provides a novel and improved fiber optic device in which an elongated handpiece for an optical fiber defines an internal, elongated, confined flow passageway with an apertured endwall at a distal end portion of the passageway, and controls the length of optical fiber that can pass through the handpiece. An optical fiber having a distal working end is dispensed through the handpiece and is disposed within the passageway. A stop member, such as a rigid sheath and the like, within the passageway surrounds and is attached to the optical fiber. The stop member limits the length of optical fiber that can pass through the handpiece. The stop member preferably contains a series of index positions for incrementally adjusting the position of the stop member within the passageway and thus the position of the optical fiber working end. The stop member is configured to abut the endwall when the stop member is moved within the internal passageway to a final position.

Preferably, a tube or cannula extends from the distal end of the elongated handpiece and defines an extension of the passageway through which the optical fiber passes. The working end of the optical fiber extends beyond the free end of the cannula in such a case. After an initial use, the stop member and the optical fiber are advanced a predetermined distance, and the portion of the free end of the optical fiber, damaged during the prior lasing procedure, is removed so as to expose a new working end. Spaced markings may be provided on the optical fiber to indicate the amount of optical fiber advanced within the internal passageway and available for use.

### BRIEF DESCRIPTION OF THE DRAWINGS

In the drawings,

FIG. 1 is a diagrammatic representation of a system employing a first embodiment of a fiber optic device illustrating certain aspects of the present invention;

FIG. 2 is a fragmentary cross-sectional view of the fiber optic device, taken along the plane 2-2 of FIG. 1;

FIG. 3 is a fragmentary cross-sectional view of a second embodiment of a fiber optic device illustrating certain aspects of the present invention;

FIG. 4 is a fragmentary cross-sectional view of the fiber optic device of FIG. 3, shown ready for last use;

FIG. 5 is a fragmentary cross-sectional view of the fiber optic device of FIG. 4, shown after its last use;

FIG. 6 is a fragmentary top plan view of a third embodiment of a fiber optic device illustrating certain aspects of the present invention;

FIG. 7 is a fragmentary cross-sectional view of a fourth embodiment of a fiber optic device illustrating certain aspects of the present invention;

FIG. 8 is a fragmentary cross-sectional view of a fifth embodiment of a fiber optic device illustrating certain aspects of the present invention;

FIG. 9 is a fragmentary cross-sectional view of a sixth embodiment of a fiber optic device illustrating certain aspects of the present invention;

FIG. 10 is a fragmentary cross-sectional view of a seventh embodiment of a fiber optic device illustrating certain aspects of the present invention;

FIG. 11 is a fragmentary cross-sectional view of components employed in the embodiment of FIG. 10;

FIG. 12 is a fragmentary cross-sectional view of an eighth embodiment of a fiber optic device illustrating certain aspects of the present invention; and

FIG. 13 is a fragmentary cross-sectional view of a ninth embodiment of a fiber optic device illustrating certain aspects of the present invention.

### DESCRIPTION OF THE PREFERRED EMBODIMENTS

For ease of description, fiber optic devices embodying the present invention are described herein below in their usual assembled position as shown in the accompanying drawings and terms such as front, rear, upper, lower, horizontal, longitudinal, distal, proximal, etc., may be used herein with reference to this usual position. However, the fiber optic devices may be manufactured, transported, sold, or used in orientations other than that described and shown herein.

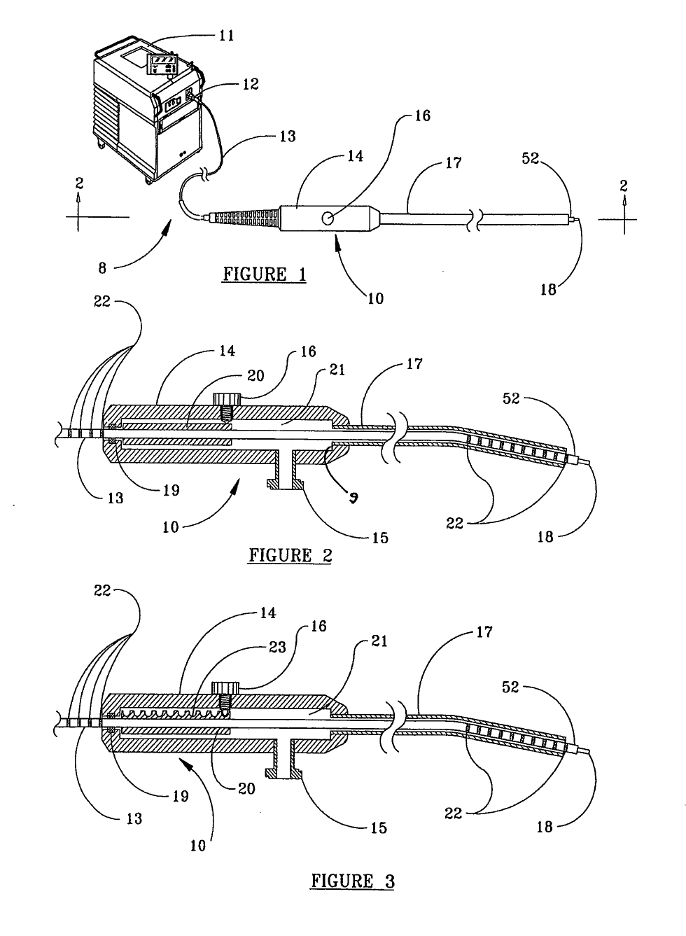

Referring now to the drawings and initially to FIGS. 1 and 2, a first embodiment is shown in relation to system 8 for delivering laser energy. As seen in FIG. 1, system 8 is comprised of a fiber optic device 10 and a source of laser energy 11. Fiber optic device 10 comprises optical fiber 13, handpiece 14, and a connector 12 that optically couples optical fiber 13 to the source of laser energy 11. Optical fiber 13 has core 18 surrounded by a buffer coating 52, and extends through an optional rubber or plastic strain relief, as known in the art, and through an enclosing body or handpiece 14. A fiber advancement mechanism is associated therewith. Handpiece 14 is preferably made of a sturdy metal, such as medical grade stainless steel, or a rigid, durable plastic, such as acetal homopolymer resin, available from Interstate Plastics, Sacramento, Calif. Handpiece 14 is comprised of two halves, which can be joined together with a locking mechanism, screws and/or an adhesive.

Optical fiber 13 extends through an elongated hollow passageway 21 (FIG. 2) defined by handpiece 14 and a tubular extension thereof which can be unitary with the handpiece 14 or a tube segment extending therefrom, such as cannula 17. If it is desired to infuse a fluid into the passageway in handpiece 14, optionally, a “luer” fitting 15, as known in the art, may extend into and provide a fluid infusion port in confined flow fluid communication with the passageway through handpiece 14.

A portion of optical fiber 13 is encased in a sturdy, rigid sheath 20 (FIG. 2) which is moveably disposed within the passageway 21 in handpiece 14. Sheath 20 serves as a stop member for the optical fiber, and may be made of metal, such as medical grade stainless steel. Sheath 20 may also be made of a biocompatible, rigid plastic, such as polyetheretherketone (PEEK) tubing, or a biocompatible, semi-rigid plastic, such as fluorocarbon, nylon or polyether block amide (PEBA) tubing, all of which are commercially available from Zeus Industrial Products, Orangeburg, S.C. Sheath 20 may have a round, square or rectangular cross-section or any other shape, preferably a square or rectangular cross-section. Passageway 21 in the handpiece 14 has a slightly larger cross-section, but of the same cross-sectional shape as sheath 20, to enable sheath 20 to move along the passageway.

With reference to FIG. 2, an indexing member such as threaded set screw 16 terminating in a tip that can be screwed down to removably fix the above mentioned sheath 20 encasing optical fiber 13 in place, at a fixed position within passageway 21 in handpiece 14. If handpiece 14 is made of a plastic, a metal insert with a matching threaded bore hole may optionally be incorporated in handpiece 14 at the time of its manufacture, to provide a stronger anchorage for set screw 16. Sheath 20 has sufficient density and thickness to prevent damage to the optical fiber from the set screw and its tip.

Distal end portion of optical fiber 13 extends through cannula 17, whose proximal end is mounted to the handpiece 14 and preferably fixed within the apertured endwall of handpiece 14 by an adhesive or other means, as known in the art, so as to form a portion of the fiber optic device 10. Cannula 17 preferably is a hollow and rigid tube, but may be flexible, semi-rigid or malleable, especially if the intended use is in conjunction with an instrument channel of a flexible endoscope. Cannula 17 may be made of a metal material, such as medical grade stainless steel, a biocompatible, rigid plastic, such as PEEK tubing, a biocompatible, semi-rigid or flexible plastic, such as fluorocarbon, nylon or polyether block amide tubing, and the like. Cannula 17 is in fluid communication with handpiece 14 and defines a continuation of passageway 21. While cannula 17 may be straight, a portion of its distal end may optionally be bent at an angle as shown in FIG. 2 to facilitate directing laser energy to a target tissue site. Cannula 17 typically is provided as part of fiber optic device 10, but it may be provided separately, as an accessory, if desired.

Optical fiber 13 has a working distal end that extends beyond the distal end of cannula 17. As illustrated, a portion of the plastic buffer coating 52 has been removed from the distal or working end of optical fiber 13, exposing the bared, glass clad core 18 of optical fiber 13. By way of an example of its use, fiber optic device 10 of the present invention may be typically supplied by the manufacturer with, for example, about 4 mm of buffer coating 52 extending distally from the distal end of the cannula and about 4 mm of the bare, glass cladding covered core 18, optical fiber 12 extending from the distal end of buffer coating 52. During its first use, the exposed core 18 of optical fiber 13 may be deformed and the distal end of protective buffer coating 52 may be degraded, from both laser energy reflected from the target tissue and the hot gasses created by the vaporization of tissue. Accordingly, ongoing maintenance procedures must be performed on the optical fiber device if it is to be reused. To that end, an additional portion of buffer coating 52 and core 18 of optical fiber are advanced or dispensed through handpiece 14, providing a new working end.

Referring to FIG. 2, optical fiber 13 extends through snugly fitting gasket 19 mounted in the proximal end of handpiece 14 to prevent leakage of fluid from the proximal end of handpiece 14, as known in the art. Optical fiber 13 extends through and is affixed by an adhesive or the like within sturdy, rigid metal or plastic sheath 20. Sheath 20 may have a round, square, rectangular or other shaped cross-section, preferably square or rectangular. Passageway 21 within handpiece 14 has slightly larger cross-sectional dimensions compared to those of sheath 20, and preferably has the same cross-sectional shape.

Set screw 16 may have a rounded, pointed, flat or other shaped distal end or tip, preferably pointed, as shown. When set screw 16 is screwed into handpiece 14 and tightened-down upon sheath 20, set screw 16 removably fixes sheath 20 and attached optical fiber 13 in place within handpiece 14.

Cannula 17 is preferably made of metal, such as medical grade stainless steel, but may also be made of a biocompatible, rigid plastic, such as PEEK tubing, a biocompatible semi-rigid plastic, a flexible plastic, and the like. If cannula 17 is made of a rigid metal or rigid plastic, the distal end portion of cannula 17 may be bent downward from its axis, as shown, at an angle of about 10° or more to better address the target tissue. A bend greater than about 10° to 15° of a rigid metal or plastic, although desirable, may not be feasible, as the bend may be too great to enable cannula 17 to pass through the instrument channel of a conventional endoscope or cystoscope. If cannula 17 is made of a semi-rigid plastic, its distal end portion may be bent at a substantially greater angle.

Spaced markings 22 may be provided on optical fiber 13, either adjacent the proximal end of handpiece 14 and/or distally from the distal end of cannula 17 (as shown for example, at the left hand and/or right hand end of FIG. 2). Markings 22 are preferably cylindrical or arcuate, and preferably are spaced apart from each other by the distance that optical fiber 13 is intended to be advanced after each use. Markings 22 may partially surround optical fiber 13, and indicia such as numbers (not separately shown) may appear between markings 22 to indicate to the operator the number of the present use or the number of uses remaining. Accordingly, when a new marking 22 is made to appear at the proximal end of handpiece 14 and/or the distal end of cannula 17, optical fiber 13 has been advanced the proper distance for another reuse, and set screw 16 can be tightened down to removably fix sheath 20 and attached optical fiber 13 in place, at the new use position.

As illustrated in FIGS. 3-5, in a second embodiment of the present invention, sheath 20 has indexing capability in the form of an array of adjacent indentations 23 on its upper surface, opposite set screw 16. Set screw 16 may be provided with a rounded tip that enters one of indentations 23 to removably fix the optic fiber 13 in place within handpiece 14. Preferably the tip of set screw 16 and the indentations 23 are complementary. The number of indentations 23 in the array preferably corresponds to the number of intended uses of device 10, as described heretofore. The spaces between the centers of indentations 23 are preferably equal to the amount of intended protrusion of optical fiber 13 from the free end of cannula 17, i.e., length of advance of buffer coating 52 and core 18 of optical fiber 13 after each use. The spacing of indentations provides a convenient indexing of the optical fiber as it is readied for shortening of its free end, prior to a subsequent use. As shown in FIG. 3, the distal end of set screw 16 has been advanced to engage the initial, or most distal indentation 23 for the first use of device 10, with optical fiber 13 extended a preferable distance distally from cannula 17. A desired length of buffer coating 52 has been removed from the distal, working end of optical fiber 13, exposing a desired length of core 18.

In the illustrated embodiment of FIG. 3, cannula 17 is made of a semi-rigid plastic tubing, and its distal end portion may be bent downwardly from its central longitudinal axis during a molding, heat treating or other conventional process, to form an angle of about 20° to 40°, but preferably about 30°. Cannula 17 is temporarily straightened out by its insertion into the instrument channel of an endoscope and returns approximately to its original bent configuration upon emerging from the distal end of the endoscope to address the target tissue.

In FIG. 4, set screw 16 is shown inserted into the last indentation 23 from the distal end of sheath 20, prior to the last use of optical fiber 13. As with the other, prior uses of optical fiber 13, its free end is extended out of the distal end of cannula 17 by a suitable distance for its clipping and cleaving, to remove the prior, now damaged buffer coat 54 and core tip 56 working end of optical fiber 13, thereby exposing a sufficient length of new working end or tip of optical fiber 13 having buffer coating end portion 60 and core tip 58. As shown in FIG. 4, tip 56 of core 18 as well as distal end portion 54 of the buffer coating 52 of optical fiber 13 have been damaged during their prior use by the back scatter of laser energy from the target tissue and hot gases generated at the treatment site.

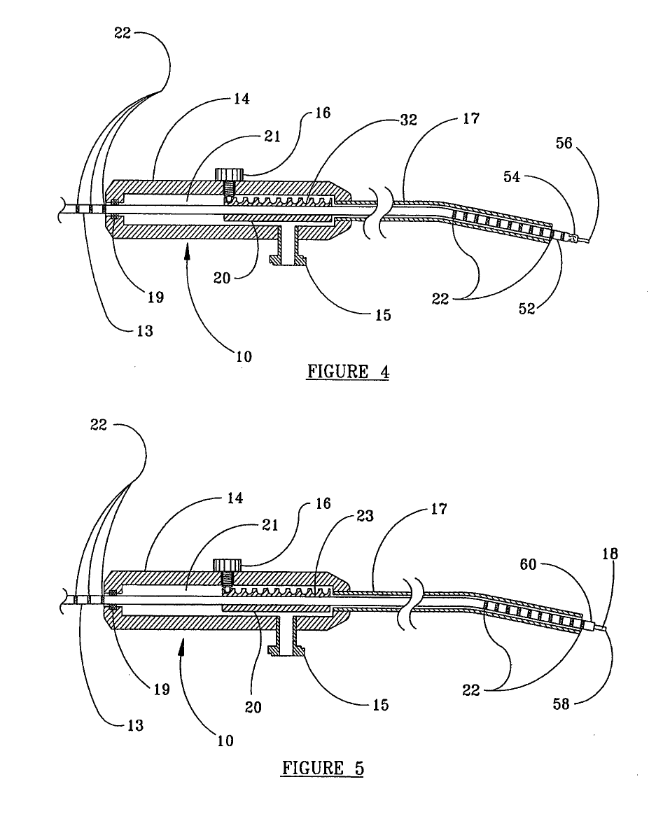

FIG. 5 illustrates device 10 ready for its final use cycle, after the damaged buffer coating end portion 54 and the tip 56 of core 18 shown in FIG. 4 have been clipped off exposing new tip 58 and buffer coating end portion 60.

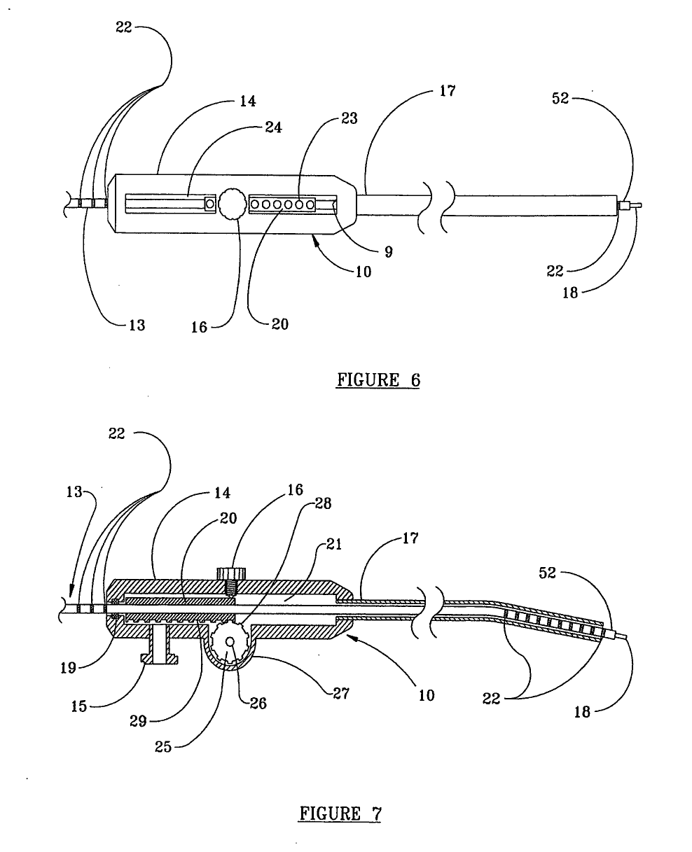

FIG. 6 illustrates a third embodiment of the present invention. In this embodiment, handpiece 14 has two viewing slots for the stop member on its top surface, one distal to expose sheath 20 that receives set screw 16, and a second, proximal slot 24. In the preferred embodiment, these slots are covered by a transparent glass or clear plastic insert and sealed with a gasket material, as known in the art, to provide a window so that sheath 20 and indentations 23 can be viewed by a user, while preventing leakage of fluid.

Optionally, slot 24 may be provided in one or both sides of handpiece 14, extending throughout most of the length of handpiece 14, to provide the functionality of the two slots shown in FIG. 6. When thus constructed, the single slot formed in handpiece 14 is filled by a glass or clear plastic insert and sealed with a gasket material as described above, to enable the operator to fully view sheath 20 and indentations 23.

In the embodiment shown in FIG. 6, sheath 20 with indentations 23 functions as an indexing device controlling advancement of fiber optic 13 beyond the distal end of cannula 17. It is generally preferred that sheath 20 be dimensioned to interfere with or abut a stop such as apertured endwall 9 at the end of the passageway within handpiece 14.

FIG. 7 illustrates a fourth embodiment of device 10. In this embodiment, gear 25 is mounted on gear shaft 26 within housing 27 of handpiece 14. Gear teeth 28 of gear 25 engage rack gear 29 preferably provided as features formed in sheath 20. Gear shaft 26 extends into a recess (not separately shown) in the bottom of housing 27 and extends through a retaining ring (not separately shown) to prevent its exiting the top of housing 27.

Shaft 26 further extends through the top of housing 27, through a snugly fitting gasket (not separately shown) that prevents leakage of fluid from the junction of shaft 26 with the top of housing 27. A knob by which gear 25 can be turned, is fixedly attached by welding or other means, as known in the art, to the end of gear shaft 26 that extends through the top of housing 27, if desired. When the gear 25 is turned clockwise, for example, gear 25 advances sheath 20 and attached optical fiber 13.

A tactile or audible mechanism (not separately shown), as known in the art, may be attached to gear 25, to advise the operator that gear 25 has advanced sheath 20 and attached optical fiber 13 to the next operating position. The sheath may then be fixed in place by set screw 16 in preparation for clipping and cleaving of optical fiber 13 prior to its next use. The diameter of gear 25 and the spacing of gear teeth 28 may be based upon the distance sheath 20 and attached optical fiber 13 are to be advanced by one or more clicks to its next clipping and cleaving portion.

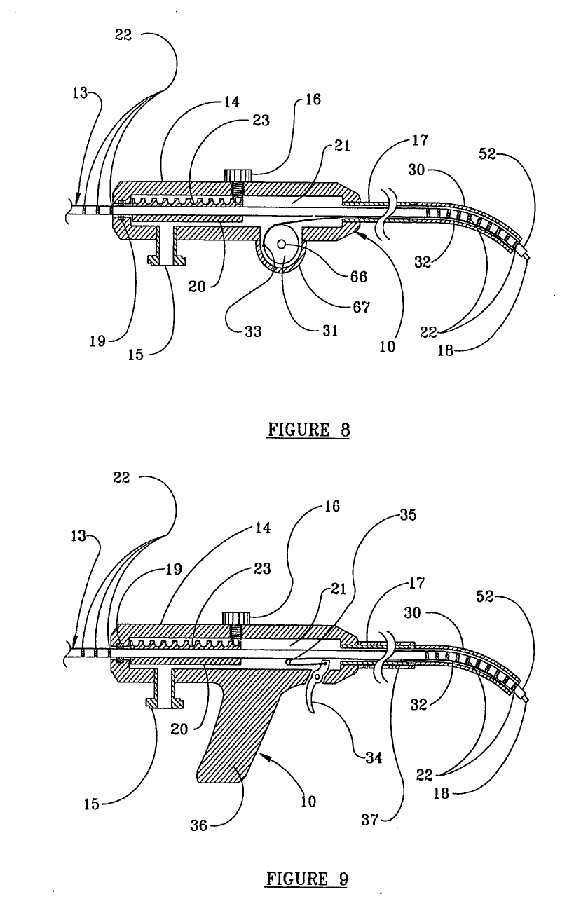

FIG. 8 illustrates a fifth embodiment of device 10 wherein an adjustable curvable tip for supporting the free end of optical fiber 13 is provided. In this embodiment, flexible, biocompatible plastic tube 30 extends distally from the distal end of cannula 17. The proximal end of flexible plastic tube 30 may be attached to the distal end of cannula 17 by an adhesive, an adhesively attached collar over the junction of cannula 17 and plastic tube 30, or other means, with the distal end portion of cannula 17 optionally overlapping the proximal end portion of flexible tube 30 or vice versa, as known in the art.

Wire 32 is anchored to the distal end of flexible tube 30 and is manipulated by wheel 31 in housing 67. The proximal end of wire 32 is force-fit into and/or fixed by welding, solder or other means, as known in the art, in a pocket or depression 33 in wheel 31. Shaft 66 of wheel 31 extends into a recess in the bottom of housing 67 and extends through a retaining ring (not separately shown) for the same purpose as described in FIG. 7, and through the top of housing 67, through a snugly fitting gasket (not separately shown) to prevent leakage of fluid from housing 67.

A knob (not separately shown), can be fixedly attached by welding or other means to the end of shaft 66 extending through the top of housing 67, to turn wheel 31, causing wire 32 to be tightened and flexible plastic tube 30 to be bent at a desired angle to better address the target tissue.

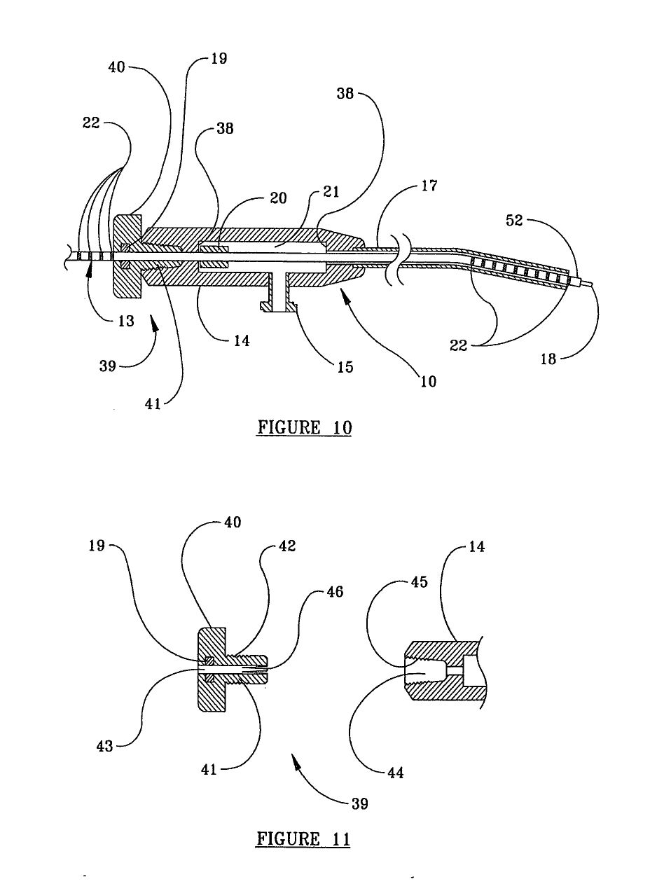

FIG. 9 shows a sixth embodiment of fiber optic device 10. This embodiment preferably comprises a variation of the embodiment of FIG. 8, in which the proximal ends of wire 32 is fixedly attached to lever 34, whose distal end portion is made in the shape of a trigger (as shown). The distal end portion of lever 34 can also be made in the shape of a hollow ring or any other desired shape. Wire 32 passes around rod 35 and its distal end is fixedly attached to the distal end of flexible tube 30, as described in FIGS. 7 and 8. When handle 36 is gripped and lever 34 is moved toward handle 36, wire 32 causes flexible tube 30 to be bent at a desired angle. One or any number of wires 32 may be used. Other variations for tensioning the wires so as to bend flexible tube 30, as are known in the art, may also be used. If desired, a geared rack can be employed in place of lever 34.

Optionally, hollow tube 30 can be made of a flexible memory metal, such as nitinol, a composition of 45% nickel and 55% titanium, made by Memry, Inc. of Bethel, Conn., and can be attached to metal or plastic cannula 17 by an adhesive and/or crimping or other means, as known in the art. The distal end portion of hollow tube 30 can be bent at a desired angle, preferably about 10 to 40 degrees or more, and made to conform to its bent shape by thermal treating. After being straightened-out, while confined, for example, in the instrument channel of an endoscope, hollow tube 30 returns to its original bent shape when it emerges from the endoscope.

Alternatively, hollow tube 30 may be made of a memory metal or plastic which has been heat treated and forced to assume a bent shape at a temperature of, for example, greater than abut 30° C. and a straight shape when cooled to a temperature less than about a 30° C. transition temperature. For example, tube 30 may be cooled in a bath of sterile, cold water and inserted into an endoscope, body orifice or surgical pathway. When it reaches body temperature, it resumes its bent shape to better address the target tissue. After use, when cooled by the infusion of sterile, cold water through cannula 17, hollow tube 30 resumes its straight shape for removal from the endoscope, body orifice or surgically created passageway.

Referring again to FIG. 9, metal cannula 17 may optionally be covered by plastic sleeve 37, preferably a lubricious plastic such as a fluorocarbon, e.g., Teflon® PTFE tubing, available from Zeus Industrial Products, Inc., Orangeburg, S.C., to ease its insertion into and through the instrument channel of an endoscope, body orifice or surgically created passageway. Sleeve 37 can be attached to cannula 17 by heat-shrinking, an adhesive or other means known in the art.

If flexible tube 30 is made of a flexible memory metal, as described above, it is also preferably covered by a lubricious plastic sleeve 37 to both ease its insertion into and through the instrument channel of an endoscope and to prevent damage to the instrument channel's surface during its advancement and withdrawal.

A seventh embodiment is illustrated in FIG. 10, with a hollow sheath 20, of cylindrical, square, triangular, rectangular or other cross-sectional shape affixed to optical fiber 13. The outside dimensions of sheath 20 are somewhat smaller than the inside dimensions of hollow passageway 21 in handpiece 14.

Sheath 20 may be attached to optical fiber 13 by an adhesive, crimping or other means, as known in the art. For simplicity and low manufacturing cost, the exterior of sheath 20 and the interior passageway 21 of handpiece 14 both preferably have a cylindrical cross-sectional shape.

Sheath 20 can be made of metal such as medical grade stainless steel, or a rigid plastic, such as PEEK tubing. In this embodiment, there is no set screw as described above, and the length of sheath 20 can be made relatively short, but with a sufficient inner surface area to enable sheath 20 to be fixedly attached to optical fiber 13 with an adhesive, as known in the art. For example, sheath 20 can be about 5 to 15 mm in length.

Sheath 20 can also be very short, to resemble a retaining ring, with a length of only about 2 to 4 mm. To assure that ring-like sheath 20 is not dislodged from optical fiber 13, sheath 20 can be crimped to optical fiber 13. However, crimping sheath 20 to optical fiber 13 may fracture the core 18 of optical fiber 13, causing device 20 to fail to function as desired and, possibly, overheating handpiece 14. The length of passageway 21 in handpiece 14, may be sized, for example, to allow the device to be used 10 times. If optical fiber 13 is to be extended for each use is 8 mm, the length of sheath 20 is 6 mm, is 10 times 8 mm plus 6 mm or 86 mm in length.

Shoulders 38 at the proximal and distal ends of channel or passageway 21 are sized so as to prevent sheath 20 from exiting passageway 21. As described heretofore, markings 22 on optical fiber 13 (located proximal to the proximal end of handpiece 14 and/or distal to the distal end of cannula 17), provide a next marking that enables the operator to position optical fiber 13 at the proper position for the next clipping and cleaving procedure of optical fiber 13, prior to the next intended use of device 10. In this embodiment, the operator tightens compression nut 41 so as to cause optical fiber 13 to be removably and sealingly fixed within handpiece 14 With this arrangement, set screw 16 of FIGS. 1-9, indentations 23 of FIGS. 3-6 and 8-9 and gear 25 and ridges 28 of FIG. 7 can be eliminated.

Referring again to FIG. 10, threaded compression assembly 39 is incorporated in the proximal end of handpiece 14 to mechanically grip optical fiber 13 and/or compress gasket 19 to removably and sealingly fix optical fiber 13 in place within handpiece 14. The benefit of this embodiment is its simplicity of construction and lower manufacturing cost, enabling device 10 to be sold at a lower price in underdeveloped countries, where the patient has to pay the cost of the medical procedure or where governmental or insurance health care programs are not readily available to pay for such devices. However, while not shown separately in this embodiment, wheel 25 and wires 31 of FIG. 8 and handle 36, trigger 34, rod 35 and wires 31 of FIG. 9 may be added to bend flexible plastic tube 30 of FIGS. 8 and 9 at a desired angle to better address the target tissue. Also, cannula 17 and/or tube 30 in this embodiment may be covered with lubricious plastic sleeve 37, as described with respect to FIG. 9.

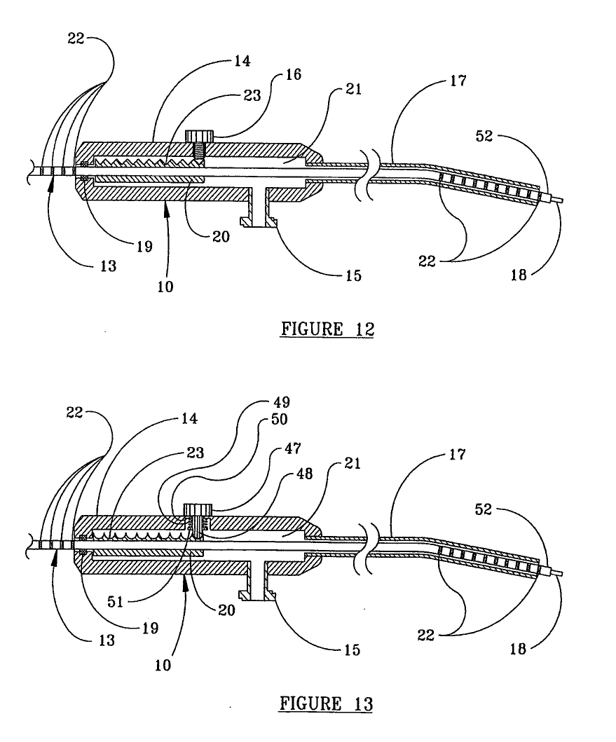

FIG. 11 illustrates components used in the construction of compression assembly 39 of FIG. 10. Nut 40 has a threaded, cylindrical nose portion 41, with threads 42 about its exterior surface. Channel 43, extending through nut 40 and nose portion 41 have an inside diameter just slightly larger than the outside diameter of optical fiber 13 (not separately shown). Optionally, gasket 19, may be incorporated in nose portion 41, as shown. Gasket 19 is sized to snugly and sealingly encase optical fiber 13 to prevent the leakage of fluid from the proximal end of handpiece 14.

Tapered opening 44 in the distal end of handpiece 14 is sized to receive nose portion 41 and progressively decreases as opening 44 extends into handpiece 14. Opening 44 in handpiece 14 has matching cylindrical threads 45 about its inner surface to engage external threads 42 of nose portion 41. When nut 40 and its nose portion 41 are threaded into opening 44 provided in handpiece 14, the progressively decreasing diameter of opening 44 forces fingers 46 in the distal end portion of nose portion 41 to removably and sealingly fix optical fiber 13 in place within handpiece 14. Compression assembly 39 can be composed of any other means, as known in the art.

FIG. 12 illustrates an eighth embodiment of the device of the present invention. To prevent the user from positioning set screw 16 down upon the shoulder between indentations 23 (shown, for example, in FIGS. 3-5, 8 and 9), the array of contiguous indentations 23 in FIGS. 12 and 13 have a relatively wide top diameter and/or center, the distance between consecutive adjacent centers being about equal to the distance optical fiber 13 is advanced out of cannula 17 after each use for clipping and cleaving, with no shoulders between indentations 23. In this embodiment, set screw 16 has a threaded shaft with a diameter up to about equal to the distance optical fiber 13 is to be advanced after each use for clipping and cleaving. The tip of set screw 16 is conical, and indentations 23 are conical of matching size and shape dimensions as the conical tip of set screw 16. Alternatively, the threaded shaft of set screw can be relatively small, about 2 to 6 mm in diameter, and a conical distal end portion, having a diameter substantially the same as that of indentations 23, may be machined as a single piece and attached to the shaft of set screw 16 by welding, matching threads and the like.

FIG. 13 illustrates a ninth embodiment, preferably constructed as a variation of the device of FIG. 12. In this embodiment, contiguous indentations 23 are hemispherical, with a top diameter and center each about equal to the distance optical fiber 13 is advanced after each use for clipping and cleaving, with no shoulders or lands therebetween. Instead of the set screw described in the preceding FIGS. 2-6 and 8-9, spring-biased lifter 47 is employed. Shaft 48 of lifter 47 has a semi-circular distal end or tip, which fits into matchingly sized and shaped, i.e., complementary, semi-circular indentations 23, with no shoulders between indentations 23. Spring 49 is biased between shoulder 50 and ring 51 of shaft 47. Ring 51 may be attached to shaft 48 of lifter 47, just proximal to the bottom of spring 49 when spring 49 is not compressed, preferably by welding or by force fitting into a semi-circular, circumferential channel (not separately shown) in shaft 48.

Shaft 48 may be attached to lifter 47, with lifter 47 and shaft 48 having matching screw threads. Alternatively, lifter 47 and shaft 48 can be machined as one part. Shaft 48 may have a diameter up to about the distance optical fiber 13 is to be advanced after each use for clipping and cleaving. Alternatively, shaft 48 can have a diameter of about 2 to 6 mm and a hemispherically shaped distal portion, whose outside diameter is substantially the same as that of indentations 23, may be formed as a part of shaft 48 or attached to shaft 48 as described above.

The distal end of shaft 48 is raised out of matching, hemispherical indentation 23 by pulling lifter 47 upwardly, against the pressure of spring 48. Optical fiber 13 is then advanced and, when lifter 47 is released, the distal semi-circular distal end of shaft 48 seats itself in the next matchingly shaped, hemispherical indentation 23.

If desired, the distal end of shaft 48 of lifter 47 can be conical and indentations 23 of this embodiment can be of a complementary conical shape, as shown in FIG. 12, or both can be of any other matching shape, as known in the art.

Instead of lifter 47, a ball and plate mechanism (not separately shown) may enable optical fiber 13 to be advanced and the distal end of shaft 48 advanced into the next matchingly shaped indentation 23.

In any of the embodiments herein, cannula 17 can be made of a rigid metal, such as medical grade stainless steel, with its distal end optionally bent downward at an angle of about 10 degrees and, optionally, covered by lubricious plastic sleeve 37. Cannula 17 can likewise be made of a biocompatible semi-rigid plastic, such as PEEK tubing, with its distal end bent downwardly at an angle of 10 to 40 degrees or more, or cannula 17 may have a rigid proximal portion to which a flexible distal end portion of a flexible plastic or flexible memory metal is attached, the latter optionally covered by a lubricious plastic sleeve.

A device embodying the present invention provides a compromise of the desires of the manufacturer and customer. The device of the present invention is comprised of an optical fiber and a handpiece with a hollow cavity or passageway through its body, through which the optical fiber moveably extends. A gasket in the proximal end of the handpiece, through which the optical fiber snugly passes, prevents a fluid injected through a port into the passageway in the handpiece from escaping from the proximal end of the handpiece. Optionally, a threaded compression device can be used to compress the optical fiber or the gasket to removably fix the optical fiber within the handpiece.

In one example the fiber optic device according to principles of the present invention is adapted for medical use through a channel of an endoscope. The length of the cannula is determined by the length of the instrument or “working channel” of the endoscope being used and the distance the cannula is to be inserted into a body for the treatment of a particular medical condition. The length of the optical fiber extending through the cannula is also determined by the length of the cannula.

In yet another embodiment of the present invention, the fiber optic device is adapted for incorporation in an endoscope designed for limited use. The stop member, such as a rigid sheath is movably disposed within a passageway defined by the endoscope handpiece and provided with a luer fitting for fluid introduction. The optical fiber extends through a fluid-tight gasket or compression fitting in the distal end of the endoscope handpiece. The stop member, such as a sheath, is secured to the optical fiber and is sized for the number of intended uses and so that the stop member abuts an internal endoscope wall at the distal end of the passageway for the last use. The limited use endoscope and the optical fiber associated therewith are discarded after the last use.

Another example of possible use of reusable fiber optic devices according to principles of the present invention will now be given. After its first use, following cleaning and sterilization of the device, for example, the optical fiber is advanced about 8 mm and the set screw is tightened-down to fix the sheath and the attached optical fiber in place. Then, about 8 mm of the buffer coating may be clipped-off by the operator from the distal end of the optical fiber, leaving about 4 mm of the buffer coating extending distally from the distal end of the cannula and about 12 mm of bared optical fiber exposed. Finally, about 8 mm of the bared optical fiber is cleaved-off, leaving about 4 mm of bared optical fiber exposed, ready for the device's next use. In this particular embodiment, the distance between the center points of the indentations is about 8 mm.

For example, if the manufacturer determines that ten uses of the device will enable the customer to obtain an acceptable “cost per case,” while preventing the optical fiber from being excessively reused, and if an average of about 8 mm of the plastic buffer coating on the distal end of the optical fiber is clipped-off after each use, the sheath should be 10 times 8 mm or 80 mm long, plus about 4 mm (allowing for about an extra 4 mm of play at the sheath's distal end) for a total length of about 84 mm. This extra length of the sheath is provided inasmuch as the distal end of the clipping device may extend up to 3 mm or more beyond the clipping location. If five uses are contemplated, for example, and the same 8 mm of the buffer coating is clipped-off after each use, the sheath should be about 5 times 8 mm, plus about 4 mm long, for a total length of about 44 mm. This assumes the nose of the clipping device extending beyond the clipping point is about 3 mm long. Any other length of the buffer coating and optical fiber may be clipped and cleaved-off, and a clipping device with a nose longer than about 3 mm may be used, with the length of the sheath adjusted accordingly.

Markings at, for example, 8 mm or other desired intervals can be made circumferentially around or on the top surface of the buffer coating of distal end portion of the optical fiber extending distally from the distal end of the cannula. When the set screw is loosened, the optical fiber is advanced for its next use and the next marking appears at the distal end of the cannula, the operator knows the set screw may then be screwed down on the sheath to hold the optical fiber in place. Also, the buffer coating can have a number within each interval to alert the operator as to the number of uses remaining available.

The sheath optionally may have indentations in its upper surface, into which the distal end of the set screw may be inserted to better removably fix the sheath and attached optical fiber in place. The number of indentations in the sheath may be equal to the number of intended uses of the optical fiber permitted by the manufacturer. In the example cited above, the centers of the indentations should be about 8 mm apart.

To avoid the likelihood that the set screw is positioned on a land or shoulder between adjacent indentations, the open end of the indentations preferably is complementary to the dimensions of the distal end of the set screw to be received therewithin, and successive indentations are contiguous to one another so that there is no land or shoulder between the indentations.

In a lengthy procedure involving the vaporization of a large volume of tissue, it may become necessary to refurbish the working tip of the device during the procedure. For example, when two or more tip refurbishments are needed to complete the procedure, the optical fiber is advanced after each such refurbishment, and the set screw reset into the next indentation after the optical fiber has been advanced.

For its first use, with the proximal end of the sheath close to the proximal end of the hollow passageway in the handpiece, the set screw is tightened-down into the first indentation from the distal end of the sheath encasing the optical fiber. After use, the device is cleaned and sterilized, the sheath and optical fiber are advanced and clipped and cleaved for its next use, as described above. This process is repeated until the device is ready for its last use.

When the last intended use of the optical fiber is reached (e.g., when the distal end of the metal sheath contacts the shoulder at the distal end of the passageway in the handpiece) the distance the optical fiber extends distally from the distal end of the cannula should be about 12 mm, based upon the above cited example, and the clipping and cleaving procedure is repeated. After its last use, the sheath and optical fiber cannot be advanced, and the device must be discarded.

As explained herein, various arrangements of fiber optic devices are contemplated by the present invention. For example, optional slots are provided in one embodiment, in the top surface or one or both side surfaces of the handpiece may be filled with a glass or clear plastic insert and sealingly fixed in place to prevent leakage of fluid. This enables the operator to see the indentations on the handpiece. Optionally, numbers on the metal sheath opposite each indentation may alert the operator as to the number of uses consumed or the number of uses remaining.

In another embodiment described herein, one side of the sheath is provided with vertical ribs or ridges and a matching ribbed gear, rotatably fixed within the handpiece, with appropriate gaskets about its shaft to prevent leakage of fluid, enables the operator to advance the sheath and optical fiber the desired distance. Preferably one notch on the ribbed wheel or gear, with a tactile feel or sound or both, advances the sheath the proper distance to enable the set screw to be screwed into the next indentation in the sheath.

In another embodiment of the present invention, the proximal portion of the delivery cannula is made of a metal, such as medical grade stainless steel, or a rigid biocompatible plastic, such as PEEK tubing, to whose distal end a section of flexible, biocompatible plastic tubing may be attached by an adhesive, covered by an adhesively attached collar or other means known in the art. One distal end portion of the cannula may overlap the proximal end portion of the flexible tubing, or vice versa.

One or more wires, affixed to the distal end of the flexible plastic tube, extend through the delivery cannula and are attached to a lever or other mechanism, preferably attached within or to the bottom surface of the handpiece. When the wire or wires are tightened, they cause the flexible tubing to bend at a desired angle to better direct the laser energy to the desired tissue.

The handpiece may have a port or luer lock, as known in the art, into the hollow passageway in the handpiece, which is in fluid communication with the delivery cannula, to enable a fluid (gas or liquid) to be infused into the passageway, pass through the cannula and exit from the distal end of the cannula to irrigate and cool the target tissue or to displace blood or other aqueous liquid from the space between the distal end of the optical fiber and the target tissue.

Displacing blood or an aqueous irrigation liquid from the space between the optical fiber and the target tissue would enable, for example, Holmium:YAG laser energy, at a wavelength of 2100 nm, which is highly absorbed by water, or any other wavelength of laser energy, which is highly absorbed by water, to be emitted during the infusion of the fluid.

For example, a small amount of carbon dioxide gas may be infused through the port to displace blood or other aqueous liquid from the space between the distal end of the optical fiber and the target tissue. If blood or aqueous liquid exists between the distal end of the optical fiber and the tissue, it would absorb a substantial amount of the laser energy and prevent the target tissue from receiving all or most of the laser energy, as described in co-owned U.S. Pat. No. 6,953,458 B2.

While this invention is susceptible of embodiment in many different forms, there are shown in the drawings and described in detail herein specific embodiments thereof, with the understanding that the present disclosure is to be considered as an exemplification of the principles of the invention and is not to be limited to the specific embodiment illustrated.

Numerous variations and modifications of the embodiments described above can be effected without departing from the spirit and scope of the novel features of the invention. It is to be understood that no limitation with respect to the specific apparatus illustrated herein is intended or should be inferred. It is, of course, intended to cover by the appended claims, all such modifications as fall within the scope of the claims.

I claim:

1. A limited use fiber optic device comprising:
an elongated handpiece having a proximal portion and a distal portion, and defining an elongated passageway with an apertured endwall at the distal portion of the handpiece;
an optical fiber having a portion thereof slidably disposed within the elongated passageway and exiting the handpiece at the distal portion of the handpiece;
a stop member affixed to the optical fiber, movably disposed within the passageway, and configured to move along the passageway together with the optical fiber as the optical fiber is moved through the handpiece and to abut the endwall when a predetermined length of the optical fiber has passed through.

2. The fiber optic device according to claim 1 wherein the handpiece defines a viewing slot for the stop member.

3. The fiber optic device in accordance with claim 1 wherein the handpiece defines at least one viewing slot for the stop member and wherein the viewing slot is covered by a transparent insert.

4. The fiber optic device of claim 1 wherein the stop member is a rigid sheath around a portion of the optical fiber situated in the elongated passageway.

5. The fiber optic device according to claim 2 further comprising spaced markings on the optical fiber.

6. The fiber optic device according to claim 2 further comprising indexing positions on the rigid sheath for engaging an indexing member on the handpiece.

7. The fiber optic device according to claim 4 wherein the indexing portions are an array of contiguous depressions defined by the rigid sheath.

8. The fiber optic device in accordance with claim 5 wherein the elongated handpiece is provided with a set screw having a tip and each of said contiguous depressions is complementary to said tip of the set screw.

9. The fiber optic device in accordance with claim 2 further comprising a tube attached to the distal end of the elongated handpiece, further defining the elongated passageway, and enclosing a portion of the optical fiber.

10. The fiber optic device in accordance with claim 9 wherein the handpiece defines a fluid infusion port in fluid communication with the passageway.

11. The fiber optic device in accordance with claim 2 wherein the tube is rigid.

12. The fiber optic device in accordance with claim 2 wherein at least a portion of the tube is flexible.

13. The fiber optic device in accordance with claim 2 wherein at least a portion of the tube is malleable.

14. The fiber optic device in accordance with claim 2 wherein the tube is semi-rigid.

15. The fiber optic device according to claim 1 further comprising:
a tube enclosing the optical fiber, having a proximal end attached to the handpiece at the distal portion of the handpiece and a flexible distal portion terminating in an open distal end;
a selectable tensioning member supported by the handpiece;
at least one wire affixed at one end to the distal end of the flexible tube, and having a second end attached to the tensioning member; and
the selectable tensioning member being adjustable between a first position for relaxing the at least one wire and a second position for tensioning the at least one wire to cause the flexible tube and the optical fiber to bend.

16. The fiber optic device according to claim 15 wherein the tensioning member comprises a rotatable wheel.

17. The fiber optic device according to claim 15 wherein the selectable tensioning member comprises a lever.

18. A system for delivering laser energy, comprising:
a source of laser energy;
a handpiece defining an elongated internal passageway with an apertured endwall at the distal end portion of the handpiece;
a hollow tube extending distally from the apertured endwall and providing a continuation of said passageway;
an optical fiber movably disposed within the passageway, having a proximal end coupled to the source of laser energy and a distal working end extending from the hollow tube;
a rigid sheath secured to the optical fiber and movable within the internal passageway portion defined by the handpiece;
an indexing member releasably engaging said rigid sheath;
the rigid sheath being configured to abut the endwall when a predetermined length of the optical fiber has been dispensed via the internal passageway.

19. The system according to claim 18 wherein the sheath defines an array of adjacent indentations and wherein the indexing member is a set screw carried by the handpiece and adapted to engage the indentations one at a time.

20. The system according to claim 18 wherein the sheath defines an array of successive contiguous indentations and wherein the indexing member is a set screw carried by the handpiece and adapted to engage the indentations one at a time.

21. The system according to claim 18 wherein the indentations have centers spaced from one another at a distance which is substantially the same as the length of the optical fiber to be advanced after each use.

22. The system according to claim 18 wherein the indexing member is a set screw terminating in a tip and carried by the handpiece, and wherein the tip of the set screw fills the indentation when the set screw has engaged the indentation.

23. The system according to claim 18 further comprising spaced markings on the optical fiber at the distal end portion thereof.

24. The system according to claim 18 further comprising spaced markings on the optical fiber adjacent to the proximal end of the handpiece.

25. The system according to claim 18 further comprising an indexing position on the rigid sheath complementary to the indexing member while the rigid sheath is situated at a fixed, predetermined location within the passageway.

26. The system according to claim 18 wherein the handpiece defines a fluid infusion port in fluid communication with the passageway.

27. The system according to claim 18 wherein the handpiece defines a viewing slot for the stop member.

28. The system according to claim 18 wherein the handpiece defines at least one viewing slot for the stop member and wherein the viewing slot is covered by a transparent insert.

29. The system according to claim 18 further comprising:
a selectable tensioning member supported by the handpiece;
at least one wire affixed at one end to the distal end of the tube, and having a second end attached to the selectable tensioning member;
the hollow tube having a flexible distal portion that terminates in an open distal end; and
the selectable tensioning member being adjustable between a first position for relaxing the at least one wire and a second position for tensioning the at least one wire to cause the flexible distal portion of the tube and an optical fiber portion therewithin to bend.

30. The fiber optic device according to claim 29 wherein the tensioning member comprises a rotatable wheel.

31. The fiber optic device according to claim 29 wherein the selectable tensioning member comprises a lever.
